(19) United States
(12) Patent Application Publication
(10) Pub. No.: US 2019/0046536 A1
(43) Pub. Date: Feb. 14, 2019

(54) Combination Therapy

(71) Applicant: Eli Lilly and Company, Indianapolis, IN (US)

(72) Inventors: Ronald Bradley DeMattos, Zionsville, IN (US); Dustin James Mergott, Indianapolis, IN (US); John Randall Sims, II, Zionsville, IN (US)

(21) Appl. No.: 16/076,733

(22) PCT Filed: Mar. 8, 2017
(86) PCT No.: PCT/US2017/021347

§ 371 Date: Aug. 9, 2018

Related U.S. Application Data
(60) Provisional application No. 62/309,016, filed on Mar. 16, 2016.

(51) Int. Cl. A61K 31/542; C07K 16/18; A61K 39/395

(52) U.S. Cl. CPC A61K 31/542; A61K 2039/505; A61K 39/3955; C07K 16/18

(57) ABSTRACT

The present invention provides a method of treating a cognitive or neurodegenerative disease, comprising administering to a patient in need of such treatment an effective amount of a compound of the formula: or a pharmaceutically acceptable salt thereof in combination with an effective amount of an anti-N3pGlu Abeta antibody selected from the group consisting of hE8L, Antibody I, and Antibody II.

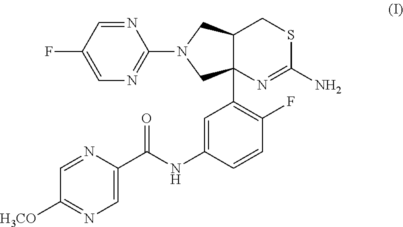 [SMILES: COc1cnc(C(=O)Nc2ccc(F)c([C@]34CN(c5ncc(F)cn5)C[C@H]3CSC(N)=N4)c2)cn1]

The present invention relates to a combination of a BACE inhibitor with an anti-N3pGlu Abeta antibody, and to methods of using the same to treat certain neurological disorders, such as Alzheimer's disease.

The present invention is in the field of treatment of Alzheimer's disease and other diseases and disorders involving amyloid β (Abeta) peptide, a neurotoxic and highly aggregatory peptide segment of the amyloid precursor protein (APP). Alzheimer's disease is a devastating neurodegenerative disorder that affects millions of patients worldwide. In view of the currently approved agents on the market which afford only transient, symptomatic benefits to the patient, there is a significant unmet need in the treatment of Alzheimer's disease.

Alzheimer's disease is characterized by the generation, aggregation, and deposition of Abeta in the brain. Complete or partial inhibition of beta-secretase (beta-site amyloid precursor protein-cleaving enzyme; BACE) has been shown to have a significant effect on plaque-related and plaque-dependent pathologies in mouse models. This suggests that even small reductions in Abeta peptide levels might result in a long-term significant reduction in plaque burden and synaptic deficits, thus providing significant therapeutic benefits, particularly in the treatment of Alzheimer's disease.

Moreover, antibodies that specifically target N3pGlu Abeta have been shown to lower plaque level in vivo (U.S. Patent Application Publication No. 2013/0142806). These antibodies are referred to herein as “anti-N3pGlu Abeta. N3pGlu Abeta, also referred to as N3pGlu Aβ, N3pE or Abetaₚ₃₋₄₂, is a truncated form of the Abeta peptide found only in plaques. Although N3pGlu Abeta peptide is a minor component of the deposited Abeta in the brain, studies have demonstrated that N3pGlu Abeta peptide has aggressive aggregation properties and accumulates early in the deposition cascade.

A combination of a BACE inhibitor with an antibody that binds N3pGlu Abeta peptide is desired to provide treatment for Abeta peptide-mediated disorders, such as Alzheimer's disease, which may be more effective than either drug alone. For example, treatment with such combination may allow for use of lower doses of either or both drugs as compared to each drug used alone, potentially leading to lower side effects while maintaining efficacy. It is believed that targeting the removal of deposited forms of Abeta with an anti-N3pGlu Abeta antibody and a BACE inhibitor will facilitate the phagocytic removal of pre-existing plaque deposits while at the same time reduce or prevent further deposition of Abeta by inhibiting the generation of Abeta.

U.S. Pat. No. 8,278,334 discloses a method of treating a cognitive or neurodegenerative disease comprising administering a substituted cyclic amine BACE-1 inhibitor with an anti-amyloid antibody. J. Neuroscience, 34(35), pages 11621-11630 (2014) discloses that combined treatment with a BACE inhibitor and an anti-Abeta antibody Gentenerumab enhances amyloid reduction in APP_{London} mice. In addition, R. DeMattos, et. al., disclosed at the 2015 Alzheimer's Association International Conference (July 18-23; abstract ID No. 6350) an investigation of dose-responses and longitudinal effects of combination therapy with a plaque specific Abeta antibody (N3pG) and BACE inhibitor in aged PDAPP transgenic mice.

Accordingly, the present invention provides a method of treating a cognitive or neurodegenerative disease, comprising administering to a patient in need of such treatment an effective amount of a BACE inhibitor in combination with an effective amount of an anti-N3pGlu Abeta antibody.

More specifically, the present invention provides a method of treating a cognitive or neurodegenerative disease, comprising administering to a patient in need of such treatment an effective amount of a compound of formula:

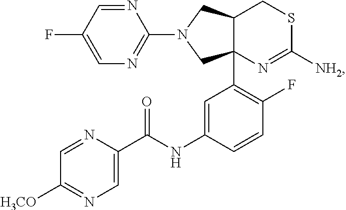 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody selected from the group consisting of hE8L, Antibody I, and Antibody II. The present invention also provides a method of treating a disease that is characterized by the formation and deposition of Abeta, comprising administering to a patient in need of such treatment an effective amount of a compound of formula:

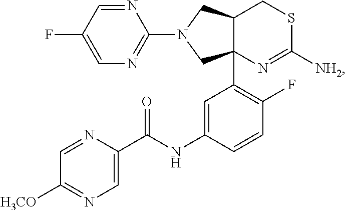 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody selected from the group consisting of hE8L, Antibody I, and Antibody II. The present invention further provides a method of treating Alzheimer's disease, comprising administering to a patient in need of such treatment an effective amount of a compound of formula:

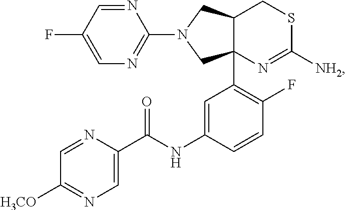 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody selected from the group consisting of hE8L, Antibody I, and Antibody II. The present invention also provides a method of treating mild Alzheimer's disease, comprising administering to a patient in need of such treatment an effective amount of a compound of formula:

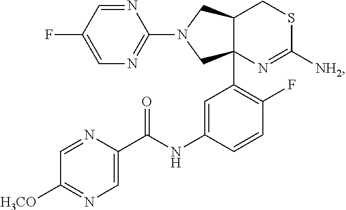 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody selected from the group consisting of hE8L, Antibody I, and Antibody II. The present invention further provides a method of treating mild cognitive impairment, comprising administering to a patient in need of such treatment an effective amount of a compound of formula:

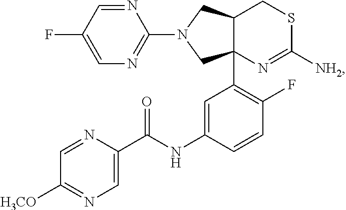 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, or a pharmaceutically acceptable salt thereof, in combination with an effective amount of an anti-N3pGlu Abeta antibody selected from the group consisting of hE8L, Antibody I, and Antibody II. The present invention further provides a method of treating prodromal Alzheimer's disease, comprising administering to a patient in need of such treatment an effective amount of a compound of formula:

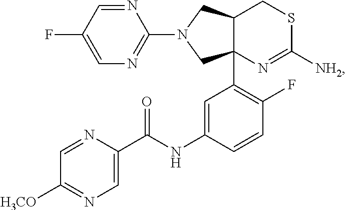 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt, in combination with an effective amount of an anti-N3pGlu Abeta antibody selected from the group consisting of hE8L, Antibody I, and Antibody II. In addition, the present invention provides a method for the prevention of the progression of mild cognitive impairment to Alzheimer's disease, comprising administering to a patient in need of such treatment an effective amount of a compound of formula:

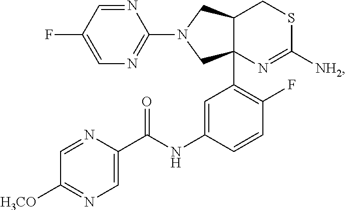 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody selected from the group consisting of hE8L, Antibody I, and Antibody II. The present invention further provides a method of treating cerebral amyloid angiopathy (CAA), comprising administering to a patient in need of such treatment an effective amount of a compound of formula:

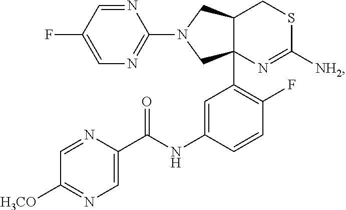 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody selected from the group consisting of hE8L, Antibody I, and Antibody II.

The present invention further provides a method of treating Alzheimer's disease in a patient, comprising administering to a patient in need of such treatment an effective amount of a compound of the formula:

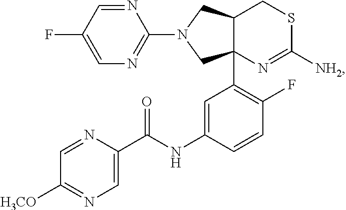 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, in combination with an effective amount of an anti-N3pGlu Abeta antibody wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 21, and HCDR3 is SEQ ID. NO: 22
b) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3; and
c) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

Furthermore, the present invention provides a compound of formula:

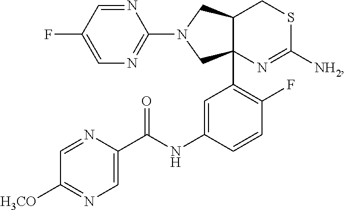 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, for use in simultaneous, separate, or sequential combination with an anti-N3pGlu Abeta antibody selected from the group consisting of hE8L, Antibody I, and Antibody II for use in the treatment of Alzheimer's disease. In addition, the present invention provides a compound of formula:

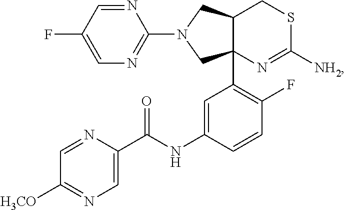 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, for use in simultaneous, separate, or sequential combination with an anti-N3pGlu Abeta antibody selected from the group consisting of hE8L, Antibody I, and Antibody II for use in the treatment of mild Alzheimer's disease. Further, the present invention provides a compound of formula:

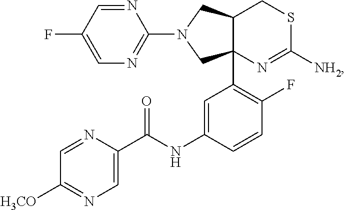 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, for use in simultaneous, separate, or sequential combination with an anti-N3pGlu Abeta antibody selected from the group consisting of hE8L, Antibody I, and Antibody II for use in the treatment of prodromal Alzheimer's disease. The present invention provides a compound of formula:

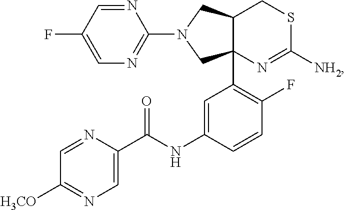 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, for use in simultaneous, separate, or sequential combination with an anti-N3pGlu Abeta antibody selected from the group consisting of hE8L, Antibody I, and Antibody II for use in the treatment of for the prevention of the progression of mild cognitive impairment to Alzheimer's disease.

The present invention provides a compound of the formula:

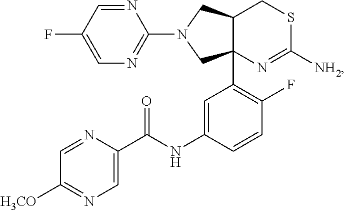 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, for use in simultaneous, separate, or sequential combination with an anti-N3pGlu Abeta wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 21, and HCDR3 is SEQ ID. NO: 22
b) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3; and
c) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3, in the treatment of Alzheimer's disease.

The invention further provides a pharmaceutical composition comprising a compound of formula:

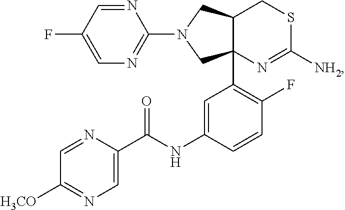 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, with one or more pharmaceutically acceptable carriers, diluents, or excipients, in combination with a pharmaceutical composition of an anti-N3pGlu Abeta antibody selected from the group consisting of hE8L, Antibody I, and Antibody II, with one or more pharmaceutically acceptable carriers, diluents, or excipients.

The invention also provides a pharmaceutical composition, comprising a compound of the formula:

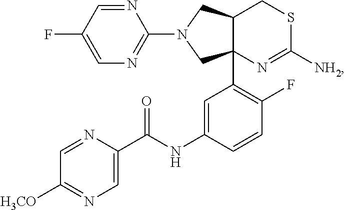 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, with one or more pharmaceutically acceptable carriers, diluents, or excipients, in combination with a pharmaceutical composition of an anti-N3pGlu Abeta antibody wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 21, and HCDR3 is SEQ ID. NO: 22
b) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3; and
c) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3, with one or more pharmaceutically acceptable carriers, diluents, or excipients.

In addition, the invention provides a kit, comprising a compound of formula:

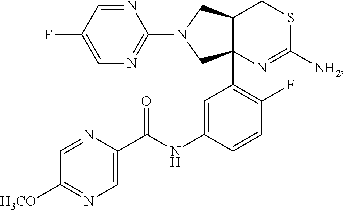 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, and an anti-N3pGlu Abeta antibody selected from the group consisting of hE8L, Antibody I, and Antibody II. The invention further provides a kit, comprising a pharmaceutical composition, comprising a compound of formula:

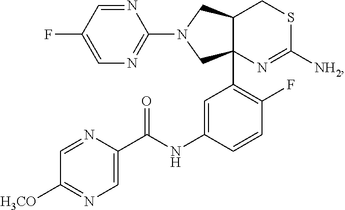 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, with one or more pharmaceutically acceptable carriers, diluents, or excipients, and a pharmaceutical composition, comprising an anti-N3pGlu Abeta antibody selected from the group consisting of hE8L, Antibody I, and Antibody II, with one or more pharmaceutically acceptable carriers, diluents, or excipients. As used herein, a “kit” includes separate containers of each component, wherein one component is a compound of formula:

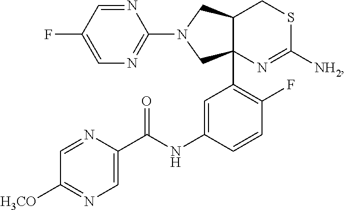 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, and another component is an anti-N3pGlu Abeta antibody selected from the group consisting of hE8L, Antibody I, and Antibody II, in a single package. A “kit” may also include separate containers of each component, wherein one component is a compound of formula:

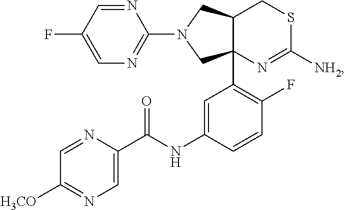 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, and another component is an anti-N3pGlu Abeta antibody selected from the group consisting of hE8L, Antibody I, and Antibody II, in separate packages with instructions to administer each component as a combination.

The invention further provides the use of a compound of formula:

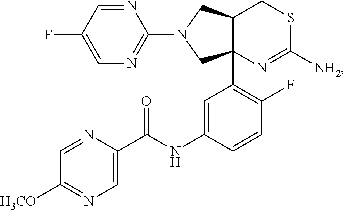 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, for the manufacture of a medicament for the treatment of Alzheimer's disease, mild Alzheimer's disease, prodromal Alzheimer's disease or for the prevention of the progression of mild cognitive impairment to Alzheimer's disease wherein the medicament is to be administered simultaneously, separately or sequentially with an anti-N3pGlu Abeta antibody selected from the group consisting of hE8L, Antibody I, and Antibody II.

The preferred antibody is hE8L.

N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide free base is a preferred compound (BACE inhibitor), and the tosylate salt of N-[3-[(4aR,7aS)-2-amino-6-(5-fluoropyrimidin-2-yl)-4,4a,5,7-tetrahydropyrrolo[3,4-d][1,3]thiazin-7a-yl]-4-fluoro-phenyl ]-5-methoxy-pyrazine-2-carboxamide is an especially preferred compound (BACE inhibitor).

The anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 21, and HCDR3 is SEQ ID. NO: 22
b) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3; and
c) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

In other embodiments, the anti-N3pGlu Abeta antibody comprises a light chain variable region (LCVR) and a heavy chain variable region (HCVR), wherein said LCVR and HCVR are selected from the group consisting of:
a) LCVR of SEQ ID NO: 23 and HCVR of SEQ ID NO: 24
b) LCVR of SEQ ID NO: 9 and HCVR of SEQ ID NO: 8; and
c) LCVR of SEQ ID NO: 10 and HCVR of SEQ ID NO: 8.

In further embodiments, the anti- N3pGlu Abeta antibody comprises a light chain (LC) and a heavy chain (HC), wherein said LC and HC are selected from the group consisting of:
a) LC of SEQ ID NO: 25 and HC of SEQ ID NO: 26;
b) LC of SEQ ID NO: 12 and a HC SEQ ID NO: 11; and
c) LC of SEQ ID NO: 13 and a HC SEQ ID NO: 11.

In further embodiments, the anti-N3pGlu Abeta antibody comprises two light chains (LC) and two heavy chains (HC), wherein each LC and each HC are selected from the group consisting of
a) LC of SEQ ID NO: 25 and HC of SEQ ID NO: 26;
b) LC of SEQ ID NO: 12 and HC of SEQ ID NO: 11; and
c) LC of SEQ ID NO: 13 and HC of SEQ ID NO: 11.

The anti-N3pGlu Abeta antibody further comprises hE8L which has a light chain (LC) and a heavy chain (HC) outlined in SEQ ID NOs: 25 and 26 respectively. hE8L further has a light chain variable region (LCVR) and a heavy chain variable region (HCVR) that are outlined in SEQ ID NOs: 23 and 24 respectively. The HCVR of hE8L further comprises HCDR1, HCDR2 and HCDR3 which are defined in SEQ ID NOs: 20, 21 and 22 respectively. Finally, hE8L further comprises LCDR1, LCDR2 and LCDR3 which are defined in SEQ ID NOs: 17, 18 and 19 respectively.

In addition, the anti-N3pGlu Abeta antibody comprises Antibody I, which has a light chain (LC) and a heavy chain (HC) outlined in SEQ ID NOs: 12 and 11 respectively. Antibody I further has a light chain variable region (LCVR) and a heavy chain variable region (HCVR) that are outlined in SEQ ID NOs: 9 and 8 respectively. The HCVR of Antibody I further comprises HCDR1, HCDR2 and HCDR3 which are defined in SEQ ID NOs: 1, 2 and 3 respectively. Finally, Antibody I further comprises LCDR1, LCDR2 and LCDR3 which are defined in SEQ ID NOs: 4, 6 and 7 respectively.

The anti-N3pGlu Abeta antibody further comprises Antibody II, which has a light chain (LC) and a heavy chain (HC) outlined in SEQ ID NOs: 13 and 11 respectively. Antibody I further has a light chain variable region (LCVR) and a heavy chain variable region (HCVR) that are outlined in SEQ ID NOs: 10 and 8 respectively. The HCVR of Antibody I further comprises HCDR1, HCDR2 and HCDR3 which are defined in SEQ ID NOs: 1, 2 and 3 respectively. Finally, Antibody I further comprises LCDR1, LCDR2 and LCDR3 which are defined in SEQ ID NOs: 4, 7 and 7 respectively.

The compound of formula:

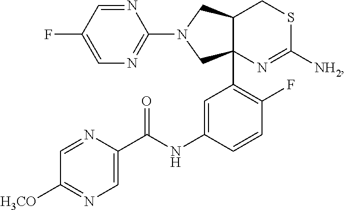 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, is disclosed as a BACE inhibitor and can be prepared by one of ordinary skill in the art as set forth in U.S. Pat. No. 8,841,293 B1, entitled “Tetrahydropyrrolothiazine Compounds”, issued Sep. 23, 2014 (U.S. Ser. No. 14/195,897); see in particular, Example 4, N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide.

One of ordinary skill in the art will further appreciate and recognize that “anti-N3pGlu Abeta antibody” and the specific antibody, “hE8L” is identified and disclosed along with methods for making and using said antibody by one of ordinary skill in the art in U.S. Pat. No. 8,679,498 B2, entitled “Anti-N3pGlu Amyloid Beta Peptide Antibodies and Uses Thereof”, issued Mar. 25, 2014 (U.S. Ser. No. 13/810,895). See for example Table 1 of U.S. Pat. No. 8,679,498 B2. The antibody, hE8L, may be used as the anti-N3pGlu Abeta antibody of the present invention. In other embodiments, the anti-N3pGlu Abeta antibody may comprise the antibody “Antibody I” described herein. In further embodiments, the anti-N3pGlu Abeta antibody may comprise “Antibody II” described herein.

In addition, amino acid sequences for certain antibodies used in the present invention are provided below in Table A:

TABLE A
Antibody SEQ ID NOs

| Antibody | Light Chain | Heavy Chain | LCVR | HCVR |
|---|---|---|---|---|
| hE8L | 25 | 26 | 23 | 24 |
| Antibody I | 12 | 11 | 9 | 8 |
| Antibody II | 13 | 11 | 10 | 8 |

With respect to “hE8L”, “Antibody I”, and “Antibody II”, additional amino acid sequences for such antibodies are provided in Table B:

TABLE B
Additional SEQ ID NOs For “hE8L”, “Antibody
I”, and “Antibody II”
Antibody SEQ ID NOs

| Antibody | LCDR1 | LCDR2 | LCDR3 |
|---|---|---|---|
| hE8L | 17 | 18 | 19 |
| I | 4 | 6 | 7 |
| II | 4 | 5 | 7 |

Antibody SEQ ID NOs

| Antibody | HCDR1 | HCDR2 | HCDR3 |
|---|---|---|---|
| hE8L | 20 | 21 | 22 |
| I and II | 1 | 2 | 3 |

The antibodies of the present invention bind to N3pGlu AP. The sequence of N3pGlu A13 is the amino acid sequence of SEQ ID NO: 27.

As used herein, an “antibody” is an immunoglobulin molecule comprising two Heavy Chain (HC) and two Light Chain (LC) interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions. Assignment of amino acids to CDR domains within the LCVR and HCVR regions of the antibodies of the present invention is based on the well-known Kabat numbering convention such as the following: Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)).

As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

Following expression and secretion of the antibody, the medium is clarified to remove cells and the clarified media is purified using any of many commonly-used techniques. The purified antibody may be formulated into pharmaceutical compositions according to well-known methods for formulating proteins and antibodies for parenteral administration, particularly for subcutaneous, intrathecal, or intravenous administration. The antibody may be lyophilized, together with appropriate pharmaceutically-acceptable excipients, and then later reconstituted with a water-based diluent prior to use. In either case, the stored form and the injected form of the pharmaceutical compositions of the antibody will contain a pharmaceutically-acceptable excipient or excipients, which are ingredients other than the antibody. Whether an ingredient is pharmaceutically-acceptable depends on its effect on the safety and effectiveness or on the safety, purity, and potency of the pharmaceutical composition. If an ingredient is judged to have a sufficiently unfavorable effect on safety or effectiveness (or on safety, purity, or potency) to warrant it not being used in a composition for administration to humans, then it is not pharmaceutically-acceptable to be used in a pharmaceutical composition of the antibody.

The term “disease characterized by deposition of Aβ,” is a disease that is pathologically characterized by Aβ deposits in the brain or in brain vasculature. This includes diseases such as Alzheimer's disease, Down's syndrome, and cerebral amyloid angiopathy. A clinical diagnosis, staging or progression of Alzheimer's disease can be readily determined by the attending diagnostician or health care professional, as one skilled in the art, by using known techniques and by observing results. This generally includes some form of brain plaque imagining, mental or cognitive assessment (e.g. Clinical Dementia Rating- summary of boxes (CDR-SB), Mini-Mental State Exam 25 (MMSE) or Alzheimer's Disease Assessment Scale-Cognitive (ADAS-Cog)) or functional assessment (e g Alzheimer's Disease Cooperative Study-Activities of Daily Living (ADCS-ADL). “Clinical Alzheimer's disease” as used herein is a diagnosed stage of Alzheimer's disease. It includes conditions diagnosed as prodromal Alzheimer's disease, mild Alzheimer's disease, moderate Alzheimer's disease and severe Alzheimer's disease. The term “pre-clinical Alzheimer's disease” is a stage that precedes clinical Alzheimer's disease, where measurable changes in biomarkers (such as CSP Aβ42 levels or deposited brain plaque by amyloid PET) indicate the earliest signs of a patient with Alzheimer's pathology, progressing to clinical Alzheimer's disease. This is usually before symptoms such as memory loss and confusion are noticeable.

As used herein, the terms “treating”, “to treat”, or “treatment”, includes restraining, slowing, stopping, reducing, or reversing the progression or severity of an existing symptom, disorder, condition, or disease.

As used herein, the term “patient” refers to a human.

The term “inhibition of production of Abeta peptide” is taken to mean decreasing of in vivo levels of Abeta peptide in a patient.

As used herein, the term “effective amount” refers to the amount or dose of compound of formula:

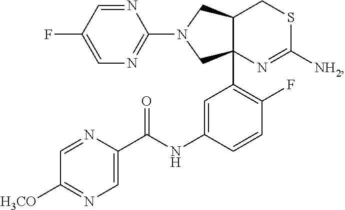 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, and to the amount or dose of an anti-N3pGlu Abeta antibody selected from the group consisting of hE8L, Antibody I, and Antibody II, which upon single or multiple dose administration to the patient, provides the desired effect in the patient under diagnosis or treatment. It is understood that the combination therapy of the present invention is carried out by administering a compound of formula:

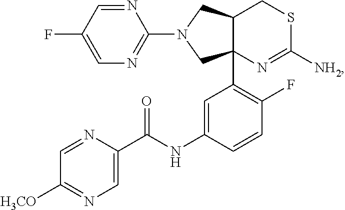 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, together with the anti-N3pGlu Abeta antibody selected from the group consisting of hE8L, Antibody I, and Antibody II, in any manner which provides effective levels of the compound of formula:

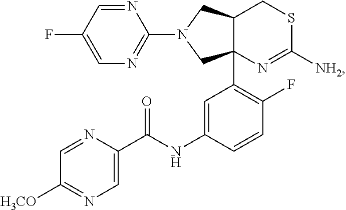 [SMILES: COc1cnc(C(=O)Nc2ccc(F)c([C@]34CN(c5ncc(F)cn5)C[C@H]3CSC(N)=N4)c2)cn1]

and the anti-N3pGlu Abeta antibody selected from the group consisting of hE8L, Antibody I, and Antibody II, in the body.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compound of formula:

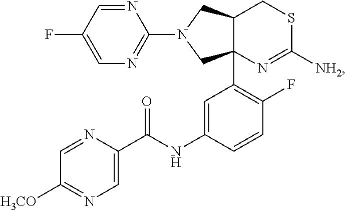 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, are generally effective over a wide dosage range in the combination of the present invention. For example, dosages per day of the compound of formula:

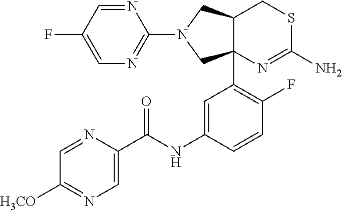 [SMILES: COc1cnc(C(=O)Nc2ccc(F)c([C@]34CN(c5ncc(F)cn5)C[C@H]3CSC(N)=N4)c2)cn1]

normally fall within the range of about 0.1 mg/day to about 500 mg/day, preferably about 0.1 mg/day to about 200 mg/day, and most preferably about 0.1 mg/day to about 100 mg/day. In some embodiments, the dose of the compound of formula:

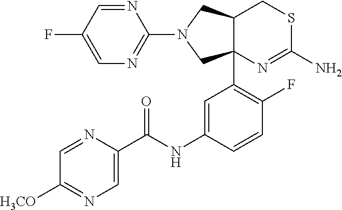 [SMILES: COc1cnc(C(=O)Nc2ccc(F)c([C@]34CN(c5ncc(F)cn5)C[C@H]3CSC(N)=N4)c2)cn1]

is about 0.1 mg/day to about 25 mg/day. In addition, the anti-N3pGlu Abeta antibody selected from the group consisting of hE8L, Antibody I, and Antibody II is generally effective over a wide dosage range in the combination of the present invention. In some instances dosage levels below the lower limit of the aforesaid ranges may be more than adequate, while in other cases still larger doses may be employed with acceptable adverse events and therefore the above dosage range is not intended to limit the scope of the invention in any way.

The BACE inhibitors and the antibodies of the present invention are preferably formulated as pharmaceutical compositions administered by any route which makes the compound bioavailable. The route of administration may be varied in any way, limited by the physical properties of the drugs and the convenience of the patient and the caregiver. Preferably, anti-N3pGlu Abeta antibody compositions are for parenteral administration, such as intravenous or subcutaneous administration. In addition, the BACE inhibitor compound of formula:

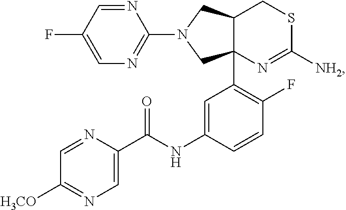 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, is for oral or parenteral administration, including intravenous or subcutaneous administration. Such pharmaceutical compositions and processes for preparing same are well known in the art. (See, e.g., Remington: The Science and Practice of Pharmacy, L. V. Allen, Editor, 22^{nd} Edition, Pharmaceutical Press, 2012).

As used herein, the phrase “in combination with” refers to the administration of the BACE inhibitor, such as the compound of formula:

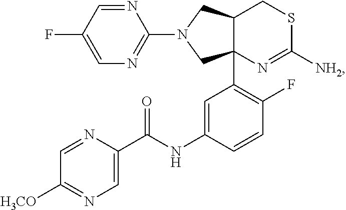 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, with an anti-N3pGlu Abeta antibody selected from the group consisting of hE8L, Antibody I, and Antibody II, simultaneously, or sequentially in any order, or any combination thereof. The two molecules may be administered either as part of the same pharmaceutical composition or in separate pharmaceutical compositions. The compound of formula:

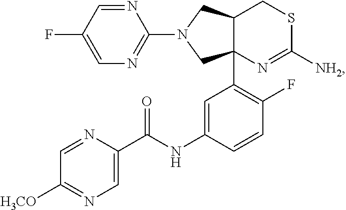 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, can be administered prior to, at the same time as, or subsequent to administration of the anti-N3pGlu Abeta antibody, or in some combination thereof. Where the anti-N3pGlu Abeta antibody is administered at repeated intervals (e.g. during a standard course of treatment), the BACE inhibitor can be administered prior to, at the same time as, or subsequent to, each administration of the anti-N3pGlu Abeta antibody, or some combination thereof, or at different intervals in relation to therapy with the anti-N3pGlu Abeta antibody, or in a single or series of dose(s) prior to, at any time during, or subsequent to the course of treatment with the anti-N3pGlu Abeta antibody.

The compounds of formula:

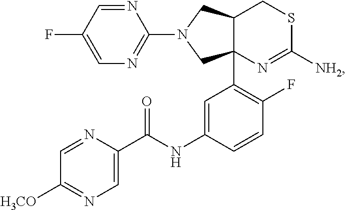 [SMILES: COc1cnc(C(=O)Nc2ccc(F)c([C@]34CN(c5ncc(F)cn5)C[C@H]3CSC(N)=N4)c2)cn1]

or pharmaceutically acceptable salts thereof, may be prepared by a variety of procedures known in the art, (see for example U.S. Pat. No. 8,841,293 B1, example 4) some of which are illustrated in the Preparations and Examples below. The specific synthetic steps for each of the routes described may be combined in different ways, or in conjunction with steps from different procedures, to prepare compounds of Formula I, or salts thereof. The products of each step can be recovered by conventional methods well known in the art, including extraction, evaporation, precipitation, chromatography, filtration, trituration, and crystallization. In addition, all substituents unless otherwise indicated, are as previously defined. The reagents and starting materials are readily available to one of ordinary skill in the art.

As used herein, “BSA” refers to Bovine Serum Albumin; “EDTA” refers to ethylenediaminetetraacetic acid; “ee” refers to enantiomeric excess; “Ex” refers to example; “F12” refers to Ham's F12 medium; “hr refers to hour or hours; “HRP” refers to Horseradish Peroxidase; “IC₅₀” refers to the concentration of an agent that produces 50% of the maximal inhibitory response possible for that agent; “min” refers to minute or minutes; “PBS” refers to Phosphate Buffered Saline; “PDAPP” refers to platelet derived amyloid precursor protein; “Prep” refers to preparation; “psi” refers to pounds per square inch; “Rₜ” refers to retention time; “SCX” refers to strong cation exchange chromatography; “THF” refers to tetrahydrofuran and “TMB” refers to 3,3′,5,5′-teramethylbenzidine.

### Example 1

### N-[3-[(4aR,7aS)-2-Amino-6-(5-fluoropyrimidin-2-yl)-4,4a,5,7-tetrahydropyrrolo[3,4-d][1,3]thiazin-7a-yl]-4-fluoro-phenyl ]-5-methoxy-pyrazine-2-carboxamide hydrochloride

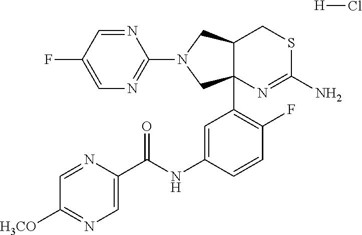 [SMILES: Cl ; COc1cnc(C(=O)Nc2ccc(F)c([C@]34CN(c5ncc(F)cn5)C[C@H]3CSC(N)=N4)c2)cn1]

N-[3-[(4aR,7aS)-2-Benzamido-6-(5-fluoropyrimidin-2-yl)-4,4a,5,7-tetrahydropyrrolo[3,4-d][1,3]thiazin-7a-yl ]-4-fluoro-phenyl]-5-methoxy-pyrazine-2-carboxamide (0.350 g, 0.58 mmol, isomer 1) is dissolved in THF (2 mL) and then methanol (4 mL) and ethanol (4 mL) are added. O-Methylhydroxylamine hydrochloride (495 mg, 5.81 mmol) and pyridine (470 μL, 5.81 mmol) are added to the mixture and the reaction is warmed to 50° C. and stirred overnight. Silica gel (˜10 g) is added to the reaction and the mixture is concentrated. The sample, dried onto silica gel, is loaded onto an empty cartridge and purified eluting with a 0-10% gradient of 7 N ammonia methanol in dichloromethane. The product is purified a second time on a SCX column using 3:1 dichloromethane:methanol and then 2:1 dichloromethane:7 N ammonia in methanol. The product is purified a final time over silica gel with a 0% to 10% gradient of 7 N ammonia methanol in dichloromethane to give the free base of the title compound. This material is dissolved in dichloromethane (5 mL) and 1 M hydrogen chloride in diethyl ether (0.20 mL, 660 μmol) is added. The solvent is removed in vacuo to give the title compound (71 mg, 23%). ES/MS (m/e): 498 (M+H).

### Example 2

### General Procedure for Preparation of Crystalline Form 2 N-[3-[(4aR,7aS)-2-Amino-6-(5-fluoropyrimidin-2-yl)-4,4a, 5,7-tetrahydropyrrolo[3,4-d][1,3]thiazin-7a-yl]-4-fluoro-phenyl]-5-methoxy-pyrazine-2-carboxamide (Hydrated)

Slurry N-[3-[(4aR,7aS)-2-amino-6-(5-fluoropyrimidin-2-yl)-4,4a,5,7-tetrahydropyrrolo[3,4-d][1,3]thiazin-7a-yl]-4-fluoro-phenyl]-5-methoxy-pyrazine-2-carboxamide in THF at about 23° C. at a concentration of about 71 mg/mL solvent. Heat the slurry with stirring to dissolution which occurs at about 60° C. to about 63° C. Add water to the hot solution to provide a THF:water solvent ratio of about 95:5. Seed crystals of Form 2 N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide are added (about 3 weight % load). The resulting thin slurry is held at about 60° C. to about 63° C. for about 20 minutes, followed by addition of about 5.3 to about 5.5 volumes of water over about 2 to about 4 hours resulting in a THF:water solvent ratio of about 69:31. The slurry is then held at about 60° C. to about 63° C. for about 30 minutes and then cooled to about 23° C. over about 1 hour, and then stirred for about 8-12 hours. The slurry is then filtered, rinsed lightly with THF:water (35:65), and dried for about 8-12 hours under reduced vacuum at about 40° C. to provide the desired crystalline Form 2 N-[3-[(4aR,7aS)-2-amino-6-(5-fluoropyrimidin-2-yl)-4,4a, 5,7-tetrahydropyrrolo[3,4-d][1,3]thiazin-7a-yl]-4-fluoro-phenyl]-5-methoxy-pyrazine-2-carboxamide which is hydrated.

### X-Ray Powder Diffraction (XRD)

The XRD patterns of crystalline solids are obtained on a Bruker D4 Endeavor X-ray powder diffractometer, equipped with a CuKa source λ=1.54060 Å) and a Vantec detector, operating at 35 kV and 50 mA. The sample is scanned between 4 and 40° in 2θ, with a step size of 0.009° in 2θ and a scan rate of 0.5 seconds/step, and with 0.6 mm divergence, 5.28 fixed anti-scatter, and 9.5 mm detector slits. The dry powder is packed on a quartz sample holder and a smooth surface is obtained using a glass slide. The crystal form diffraction patterns are collected at ambient temperature and relative humidity. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g., The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature or humidity at which a sample is analyzed, sample displacement, or the presence or absence of an internal standard. In the present case, a peak position variability of ±0.2 in 20 will take into account these potential variations without hindering the unequivocal identification of the indicated crystal form. Confirmation of a crystal form may be made based on any unique combination of distinguishing peaks (in units of ° 2θ), typically the more prominent peaks. The crystal form diffraction patterns, collected at ambient temperature and relative humidity, are adjusted based on NIST 675 standard peaks at 8.853 and 26.774 degrees 2-theta.

A prepared sample of crystalline Form 2 N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide is characterized by an XRD pattern using CuKa radiation as having diffraction peaks (2-theta values) as described in Table C below. Specifically, the pattern contains a peak at 11.8° in combination with one or more of the peaks selected from the group consisting of 18.6°, 19.3°, and 26.7°; with a tolerance for the diffraction angles of 0.2 degrees.

TABLE C
X-ray powder diffraction peaks of crystalline Form 2.

| | Angle | Relative Intensity |
|---|---|---|
| Peak | (2-Theta °) +/− 0.2° | (% of most intense peak) |
| 1 | 11.8 | 100.0 |
| 2 | 18.6 | 71.4 |
| 3 | 19.3 | 45.5 |
| 4 | 26.7 | 41.9 |
| 5 | 20.6 | 27.3 |
| 6 | 9.0 | 19.0 |
| 7 | 24.8 | 18.5 |
| 8 | 22.4 | 15.5 |
| 9 | 31.9 | 14.3 |
| 10 | 10.6 | 11.2 |

Crystalline Form 2 N-[3-[(4aR,7aS)-2-Amino-6-(5-fluoropyrimidin-2-yl)-4,4a,5,7-tetrahydropyrrolo [3,4-d][1,3]thiazin-7a-yl]-4-fluoro-phenyl]-5 -methoxy-pyrazine-2-carboxamide is a stable crystal form at room temperature and relative humidity greater than about 15%.

### Example 3

### Crystalline Form 3N-[3-[(4aR,7aS)-2-Amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide

A thermogravimetric analysis pan is loaded with N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide and heated to about 170° C. and held at 170° C. for about 5 minutes. The mixture is cooled to room temperature to provide the title compound.

### Alternative Preparation of Crystalline Form 3 N-[3-[(4aR,7aS)-2-Amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide

N-[3-[(4aR,7aS)-2-amino-6-(5-fluoropyrimidin-2-yl)-4,4a,5,7-tetrahydropyrrolo[3,4-d][1,3]thiazin-7a-yl]-4-fluoro-phenyl]-5-methoxy-pyrazine-2-carboxamide (121 mg) is combined with ACN (5 mL) in a vial, and heated on a 90° C. stir plate. After about 30 minutes, most of the solid dissolves, providing a cloudy solution. Form 3 seeds are added and the sample is stirred for about 1 hour at about 90° C. Heating is removed and the mixture is stirred to provide a bright white solid. The solid is isolated by vacuum filtration, dried under an air stream for about 10 minutes, and then under reduced vacuum at about 80° C. for about 8 to 12 hours to provide the title compound.

A prepared sample of crystalline Form 3 N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide is characterized by an XRD pattern using CuKa radiation as having diffraction peaks (2-theta values) as described in Table D below. Specifically, the pattern contains a peak at 15.7° in combination with one or more of the peaks selected from the group consisting of 18.1°, 27.0°, and 19.7°; with a tolerance for the diffraction angles of 0.2 degrees.

TABLE D
X-ray powder diffraction peaks of crystalline Form 3.

| | Angle | Relative Intensity |
|---|---|---|
| Peak | (2-Theta °) +/− 0.2° | (% of most intense peak) |
| 1 | 15.7 | 100.0 |
| 2 | 18.1 | 98.5 |
| 3 | 27.0 | 80.2 |
| 4 | 19.7 | 75.4 |
| 5 | 25.3 | 75.2 |
| 6 | 18.7 | 50.0 |
| 7 | 17.3 | 49.7 |
| 8 | 5.2 | 44.0 |
| 9 | 10.4 | 41.7 |
| 10 | 11.9 | 37.7 |

### Example 4

### Preparation of N-[3-[(4aR,7aS)-2-Amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide; toluenesulfonic acid

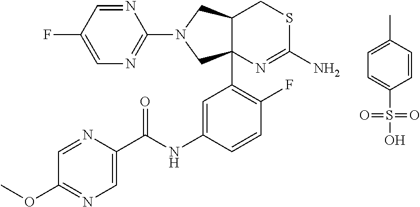 [SMILES: COc1cnc(C(=O)Nc2ccc(F)c([C@]34CN(c5ncc(F)cn5)C[C@H]3CSC(N)=N4)c2)cn1 ; Cc1ccc(S(=O)(=O)O)cc1]

Crystalline Form 2 N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide hydrated (149.15 mg) is added to ethyl acetate (2 mL). The sample is stirred at 1000 rpm at a temperature of 80° C. p-Toluenesulfonic acid (70 mg dissolved in ethyl acetate (1 mL)) is added to the stirring solution, and it is stirred overnight at 80° C. to produce a slurry of a white solid which is isolated by vacuum filtration to provide the title compound (tosylate salt).

### Alternative Preparation A of N-[3-[(4aR,7aS)-2-Amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide; toluenesulfonic acid

N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide (9.5 g, 19 mmol) and p-toluenesulfonic acid (3.80 g, 19.8 mmol) are added to tetrahydrofuran (31 mL), water (7.9 mL), and 2-propanol (8.6 mL). The solution is heated to 40° C. To the warm solution is added 2-propanol (200.0 mL) over approximately 3 hours. The mixture is seeded shortly after the start of the 2-propanol addition with a portion of the title compound (500 mg, 0.75 mmol). After the solvent addition is complete, the mixture is cooled to approximately 20° C. over 1-3 hours. The mixture is heated from approximately 20° C. to approximately 55° C. over a target time of 2 hours. The temperature is held at 55° C. for 1 hour and then cooled to about 20° C. over approximately 4 hours. The slurry is stirred for at least 10 hours at approximately 20° C. The slurry is filtered and the wet cake is washed with water (57 mL). The product is dried in vacuo at 45° C. for at least 10 hours to give the title compound (10.4 g, 81%). ES/MS (m/z): 500 (M+H).

### Alternative Preparation B of N-[3-[(4aR,7aS)-2-Amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide; toluenesulfonic acid

N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide hydrated (20.7 g) is slurried at 170 rpm in 60:40 THF:H₂O (85 mL) in a 500 mL 3-necked round bottomed flask equipped with a nitrogen bubbler, IKA® mechanical motor/agitator attached to a glass shaft having a teflon banana blade, and a thermocouple connected to a programmable J-KEM® temperature controller. p-Toluenesulfonic acid monohydrate (7.6 g, 1.03 eq) is dissolved in a mixture of 60:40 THF:H₂O (20 mL) and the solution added all at once to the stirring N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide slurry at 23° C., leading almost immediately to a clear reddish tan solution. The agitation rate is then increased to 200 rpm as over 15 minutes, water (22 mL) is added to the solution, which is then seeded with N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide toluenesulfonic acid (750 mg, 3 wt % seed load) and is then stirred at 23° C. for a further 15 minutes. Over 6 hours, water (226 mL, total solvent of 353 mL; or 13.6 vol., final solvent ratio of 17.5:82.5 THF:H₂O) is added to the slurry, which is then stirred overnight (22 hours) at 23° C. The slurry is filtered via vacuum, rinsed with 15:85 THF:H₂O (2×20 mL), then left on vacuum for 20 minutes while cracks which form in the product wet cake are manually pressed closed. The wet solids are dried at 40° C. under vacuum for about 72 hours to give the title compound as a white crystalline solid (24.07 g, 90.0 wt %).

The crystalline N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide; toluenesulfonic acid is characterized by an XRD pattern using CuKa radiation as having diffraction peaks (2-theta values) as described in Table E below, and in particular having peaks at diffraction angle 2-theta of 5.0° in combination with one or more of the peaks selected from the group consisting of 19.6°, 13.8°, and 18.5°; with a tolerance for the diffraction angles of 0.2 degrees,

TABLE E
X-ray powder diffraction peaks of crystalline Example 4

| | Angle | Relative Intensity |
|---|---|---|
| Peak | (2-Theta °) +/− 0.2° | (% of most intense peak) |
| 1 | 5.0 | 100.0 |
| 2 | 13.4 | 22.9 |
| 3 | 13.8 | 37.3 |
| 4 | 14.4 | 20.2 |
| 5 | 15.3 | 28.8 |
| 6 | 17.5 | 25.9 |
| 7 | 18.5 | 30.7 |
| 8 | 19.6 | 45.8 |
| 9 | 20.4 | 17.7 |
| 10 | 25.6 | 30.1 |

### Expression and Purification of Engineered N3pGlu A13 Antibodies

Anti-N3pGlu Aβ antibodies (Antibody I or II) of the present invention can be expressed and purified essentially as follows. A glutamine synthetase (GS) expression vector containing the DNA sequence encoding the LC amino acid sequence of SEQ ID NO: 12 or 13 and the DNA sequence encoding the HC amino acid sequence of SEQ ID NO: 11 is used to transfect a Chinese hamster ovary cell line (CHO) by electroporation. The expression vector encodes an SV Early (Simian Virus 40E) promoter and the gene for GS. Post-transfection, cells undergo bulk selection with 0-50 μM L-methionine sulfoximine (MSX). Selected bulk cells or master wells are then scaled up in serum-free, suspension cultures to be used for production.

Clarified medium, into which the antibody has been secreted, is applied to a Protein A affinity column that has been equilibrated with a compatible buffer, such as phosphate buffered saline (pH 7.4). The column is washed with 1M NaCl to remove nonspecific binding components. The bound anti-N3pGlu AP antibody is eluted, for example, with sodium citrate at pH (approx.) 3.5 and fractions are neutralized with 1M Tris buffer. Anti-N3pGlu AP antibody fractions are detected, such as by SDS-PAGE or analytical size-exclusion, and then are pooled. Anti-N3pGlu AP antibody (Antibody I or Antibody II) of the present invention is concentrated in either PBS buffer at pH 7.4 or 10 mM NaCitrate buffer, 150 mM NaCl at pH around 6. The final material can be sterile filtered using common techniques. The purity of the anti-N3pGlu Aβ antibody is greater than 95%. The anti-N3pGlu Aβ antibody (Antibody I or Antibody II) of the present invention may be immediately frozen at −70° C. or stored at 4° C. for several months.

### Binding Affinity and Kinetics

The binding affinity and kinetics of an anti-N3pGlu AP antibody (Antibody I or Antibody II) to pE3-42 Aβ peptide or to Aβ 1-40 peptide is measured by surface plasmon resonance using BIACORE® 3000 (GE Healthcare). The binding affinity is measured by capturing the anti-N3pGlu AP antibody via immobilized protein A on a BIACORE® CMS chip, and flowing pE3-42 Aβ peptide or Aβ 1-40 peptide, starting from 100 nM in 2-fold serial dilution down to 3.125 nM. The experiments are carried out at 25° C. in HBS-EP buffer (GE Healthcare BR100669; 10 mM HEPES, 150 mM NaCl, 3 mM EDTA, 0.05% surfactant P20, pH 7.4).

For each cycle, the antibody is captured with 5 μL injection of antibody solution at a 10 μg/mL concentration with 10 μL/min flow rate. The peptide is bound with 250 μL injection at 50 μL/min, and then dissociated for 10 minutes. The chip surface is regenerated with 5 μL injection of glycine buffer at pH 1.5 at 10 μL/mL flow rate. The data is fit to a 1:1 Langmiur binding model to derive kₒₙ, k_{off}, and to calculate K_{D}. Following procedures essentially as described above, the following parameters (shown in Table 2) were observed.

TABLE 2
Binding affinity and kinetics.

| | kₒₙ | k_{off} | K_{D} |
|---|---|---|---|
| Antibody | (×10⁵ 1/MS) | (×10⁻⁴ 1/s) | (nM) |
| I | 1.39 | 1.31 | 0.71 |
| II | 3.63 | 1.28 | 0.35 |

No appreciable binding to Aβ 1-40 was detected, indicating that Antibody I and Antibody II bound specifically to pE3-42 Aβ peptide as compared to Aβ 1-40.

### Ex Vivo Target Engagement

To determine ex vivo target engagement on brain sections from a fixed PDAPP brain, immunohistochemical analysis is performed with an exogenously added anti-N3pGlu Aβ antibody (Antibody I or Antibody II). Cryostat serial coronal sections from aged PDAPP mice (25-month old) are incubated with 20 μg/mL of an exemplified N3pGlu Aβ antibody of the present invention (Antibody I or Antibody II). Secondary HRP reagents specific for human IgG are employed and the deposited plaques are visualized with DAB-Plus (DAKO). Biotinylated murine 3D6 antibody followed by Step-HRP secondary is used as a positive control. The positive control antibody (biotinylated 3D6) labeled significant quantities of deposited Aβ in the PDAPP hippocampus, and the anti-N3pGlu Aβ antibodies (Antibody I or Antibody II) labeled a subset of deposits. These histological studies demonstrated that the anti-N3pGlu Aβ antibodies (Antibody I and Antibody II) engaged deposited Aβ target ex vivo.

The following Examples and assays demonstrate how a study could be designed to verify (in animal models) that the combination of antibodies of the present invention, in combination with the compound outlined herein, may be useful for treating a disease characterized by deposition of Aβ, such as of Alzheimer's disease, Down's syndrome, and CAA. It should be understood however, that the following descriptions are set forth by way of illustration and not limitation, and that various modifications may be made by one of ordinary skill in the art.

### Combination Study

### BACE Inhibitor Feeding Pilot Study

A pilot pharmacokinetic and pharmacodynamic study is performed in PDAPP mice fed a chow diet containing a BACE inhibitor, such as a compound of formula:

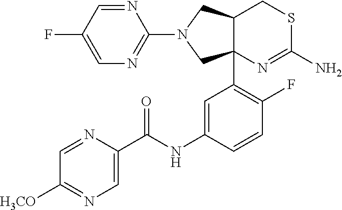 [SMILES: COc1cnc(C(=O)Nc2ccc(F)c([C@]34CN(c5ncc(F)cn5)C[C@H]3CSC(N)=N4)c2)cn1]

or pharmaceutically acceptable salt thereof, in order to define doses that provide minimal to marked plasma and brain Abeta reduction by BACE inhibition alone. Young PDAPP mice are fed for 14 days a diet containing a chow diet containing the BACE inhibitor at “quasi-bid” equivalent doses of 3 mg/kg, 10 mg/kg, 30 mg/kg, or 100 mg/kg. The BACE inhibitor at ˜0.05, 0.15, 0.5, or 1.5 mg per gram of certified rodent diet #8728CM (Harlan labs) is mixed in a Sorvall mixer for 10 minutes and then mixed with Hobart mixer for 15 minutes prior to pelleting. Thirty-two young female PDAPP mice are randomized by parental line into 4 groups of 8 consisting of a vehicle-treatment group and the three doses of BACE inhibitor. Mice are allowed ad libitum access to food for 14 days and subsequently sacrificed. Mice are anesthetized with CO₂ and blood collected by cardiac puncture into EDTA-coated microcentrifuge tubes and stored on ice. Subsequently, plasma is collected by centrifugation of blood samples for 4 minutes at 14,000 rpm at room temperature, transferred to untreated microcentrifuge tubes, then frozen on dry ice and stored at −80 ° C. until analysis. Mice are sacrificed by decapitation, brains are rapidly micro-dissected into halves, flash frozen on dry ice and stored at −80 ° C. until analysis (one half for Abeta analysis and the other half for compound exposures measurement). For analysis of parenchymal Abeta, brain samples are homogenized in 5.5 M guanidine-HCl buffer (0.5 mL per half brain) with tissue tearer (model 985-370) at speed 5 for about 1 minute. Homogenized brain samples are nutated overnight at room temperature.

For Abeta ELISA analysis, extracts are collected and diluted at least 1:10 in casein buffer (1×PBS with 0.25% casein, 0.05% Tween 20, 0.1% thimerosal, pH 7.4 with protease inhibitor cocktail (Sigma P9340 at 0.01 mg/mL)) and centrifuged at 14000 rpm for 10 minutes. For analysis of plasma Abeta, samples are diluted 1:2 in specimen buffer (PBS; 0.05% Triton X-405; 0.04% thimerasol, 0.6% BSA), prior to analysis by ELISA. Plasma human Abeta₁₋ₓ is determined by sandwich ELISA using m266.2 (anti-Abeta₁₃₋₂₈) and biotinylated 3D6 (anti-Abeta1-5) as the capture and reporter antibodies, respectively. Unknowns are assayed in duplicate and pg/mL determined by interpolating (Soft Max Pro v. 5.0.1, Molecular Dynamics, using 4-parameter fit of the reference curve) from 8 point standard curves and then adjusting for dilution. Parenchymal Abeta is determined by sandwich ELISAs as described above and the values are normalized to protein levels (determined in duplicate by the Bradford Coomassie Plus Protein method) and expressed as pg/mg protein.

To determine the tissue and plasma levels of the BACE inhibitor, the following method is employed: A 0.1 mg/mL stock solution of BACE inhibitor is serially diluted with methanol/water (1:1, v/v), to prepare working solutions, which are then used to fortify control plasma and brain homogenates to yield analyte concentrations of 1, 5, 10, 20, 50, 100, 500, 1000, 2000, 4000, and 5000 ng/mL. Prior to analysis, brain samples are homogenized in 3-volumes of methanol/water (1:4, v/v) with an ultrasonic disrupter. An aliquot of each study sample, appropriate calibration standard and control matrix samples are transferred to a 96-well plate and then mixed with acetonitrile containing internal standard. After mixing, the samples are centrifuged to pellet the precipitated proteins. Aliquots of the resulting supernatants are then transferred to a clean 96-well plate and diluted with methanol/water (1:1, v/v), and 10 microliter aliquots are analyzed by LC-MS/MS. Analyte concentrations are calculated using the response to concentration relationship determined by multiple regression of the calibration curve samples.

### In Vivo Combination Study

In order to evaluate combinational plaque lowering therapy of an anti-N3pGlu Abeta antibody such as hE8L, Antibody I or Antibody II and a BACE inhibitor, such as a compound of formula:

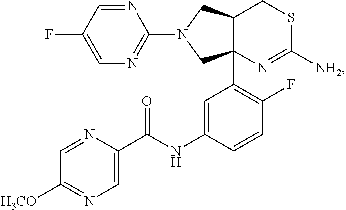 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, a large cohort of PDAPP mice are first aged to 16 to 18-months of age. The aged PDAPP mice are randomized into five treatment arms based upon gender, parental line, and age. There are 20 to 30 aged PDAPP mice per treatment arm. Group 1 is sacrificed as a time zero at study initiation in order to determine the baseline level of pathology prior to therapeutic treatment (necropsy described below). The four remaining groups are then treated as follows: Group-2, control animals receiving placebo chow diet and weekly injections of 12.5 mg/kg of control isotype IgG2a antibody; Group-3, animals receiving weekly injections of 12.5 mg/kg anti-N3pGlu-Abeta antibody; Group-4, animals receiving BACE inhibitor chow diet at doses previously defined in the pilot feeding study, but typically ˜3 to 30 mg/kg/day; Group-5, animals receiving BACE inhibitor chow diet (˜3 to 30 mg/kg/day) and weekly injections of 12.5 mg/kg of anti-N3pGlu-Abeta antibody. The anti-N3pGlu-Abeta antibody is diluted from sterile stock solutions consisting of the antibody in PBS buffer and is administered to the animals by intraperitoneal injections. The BACE inhibitor is mixed with loose chow diet (˜0.15 to 1.5 mg compound per gram of feed depending upon desired dose) and compressed into feed pellets. Animal weight is recorded at study initiation and subsequently weekly for the first month of treatment, and then monthly for the study duration. The food intake is also monitored over the course of the study at regular intervals. The animals receive the study treatments for a total of 4-months. The animals stay on their respective diets until necropsy, which occurs one week after the final antibody injections. At time of necropsy, the animals are anesthetized and blood obtained by cardiac puncture using EDTA pre-rinsed 1 ml syringes. The blood samples are collected on ice and the plasma isolated by standard centrifugation. Subsequently, the animals are perfused with cold heparinized saline and the brain removed and dissected into the left and right hemi-spheres. One brain hemi-sphere is flash frozen and saved for histological analyses. The remaining brain hemi-sphere is dissected into tissue segments consisting of hippocampus, cortex, cerebellum, and mid-brain and subsequently frozen on dry ice. The plasma and tissue samples are stored at −80° C. until time of analysis.

### Pharmacokinetic Evaluation

Plasma pharmacokinetics is determined on the plasma samples obtained at time of necropsy. Plasma antibody levels are determined in an antigen binding ELISA assay wherein plates are coated with antigen (Abetaₚ₃₋₄₂) and subsequently incubated with diluted plasma samples or a reference standard consisting of a serial dilution of the anti-N3pGlu antibody in assay buffer (PBS+control murine plasma). After washing the plate, the bound murine antibody was detected with an anti-murine-HRP conjugated antibody followed by color development with TMB. To determine the tissue (mid-brain) and plasma levels of the BACE inhibitor, the following method is employed: A 0.1 mg/mL stock solution of BACE inhibitor is serially diluted with methanol/water (1:1, v/v), to prepare working solutions, which are then used to fortify control plasma and brain homogenates to yield analyte concentrations of 1, 5, 10, 20, 50, 100, 500, 1000, 2000, 4000, and 5000 ng/mL. Prior to analysis, brain samples are homogenized in 3-volumes of methanol/water (1:4, v/v) with an ultrasonic disrupter. An aliquot of each study sample, appropriate calibration standard and control matrix samples are transferred to a 96-well plate and then mixed with acetonitrile containing internal standard. After mixing, the samples are centrifuged to pellet the precipitated proteins. Aliquots of the resulting supernatants are then transferred to a clean 96-well plate and diluted with methanol/water (1:1, v/v), and 10 microliter aliquots are analyzed by LC-MS/MS. Analyte concentrations are calculated using the response to concentration relationship determined by multiple regression of the calibration curve samples.

### Pharmacodynamic Evaluation

The parenchymal Abeta concentrations are determined in guanidine solubilized tissue homogenates by sandwich ELISA. Tissue extraction is performed with the bead beater technology wherein frozen tissue is extracted in 1 ml of 5.5 M guanidine/50 mM Tris/0.5× protease inhibitor cocktail at pH 8.0 in 2 ml deep well dishes containing 1 ml of siliconized glass beads (sealed plates were shaken for two intervals of 3-minutes each). The resulting tissue lysates are analyzed by sandwich ELISA for Abeta₁₋₄₀ and Abeta₁₋₄₂: bead beater samples are diluted 1:10 in 2% BSA/PBS-T and filtered through sample filter plates (Millipore). Samples, blanks, standards, quality control samples, are further diluted in 0.55 M guanidine/5 mM Tris in 2% BSA/PBST prior to loading the sample plates. Reference standard are diluted in sample diluent. Plates coated with the capture antibody 21F12 (anti-Abeta₄₂) or 2G3 (anti-Abeta₄₀) at 15 μg/ml are incubated with samples and detection is accomplished with biotinylated 3D6 (anti-Abeta₁₋ₓ) diluted in 2% BSA/PBS-T, followed by 1:20 K dilution NeutrAvidin-HRP (Pierce) in 2% BSA/PBS-T and color development with TMB (Pierce). The Abeta levels are interpolated from standard curves and the final tissue concentration is calculated as nanograms of Abeta per milligram of tissue wet weight. The percent area of the hippocampus and cortex occupied by deposited Abeta is determined histologically. Cryostat serial coronal sections (7 to 10 μm thick) are incubated with 10 μg/ml of biotinylated 3D6 (anti-Abeta₁₋ₓ) or negative control murine IgG (biotinylated). Secondary HRP reagents specific for biotin are employed and the deposited Abeta visualized with DAB-Plus (DAKO) Immunoreactive Abeta deposits are quantified in defined areas of interest within the hippocampus or cortex by analyzing captured images with Image Pro plus software (Media Cybernetics).

These studies may show that the combination therapy of an anti-N3pGlu-Abeta antibody, such a hE8L, Antibody I, or Antibody II, with a BACE inhibitor, such as a compound of formula:

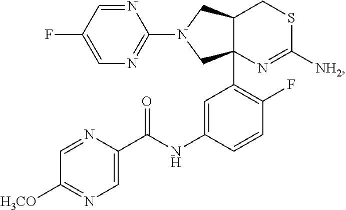 [SMILES: COc1cnc(C(=O)Nc2ccc(F)c([C@]34CN(c5ncc(F)cn5)C[C@H]3CSC(N)=N4)c2)cn1]

or a pharmaceutically acceptable salt thereof, may result in enhanced Abeta reductions relative to the individual mono-therapies.

Sequences

>SEQ ID NO: 1; PRT1; Artificial> (HCDR1-Antibody I and Antibody II)
KASGYTFTDYYIN
>SEQ ID NO: 2; PRT1; Artificial> (HCDR2-Antibody I and Antibody II)
WINPGSGNTKYNEKFKG
>SEQ ID NO: 3; PRT1; Artificial> (HCDR3-Antibody I and Antibody II)
TREGETVY
>SEQ ID NO: 4; PRT1; Artificial> (LCDR1-Antibody I and Antibody II)
KSSQSLLYSRGKTYLN
>SEQ ID NO: 5; PRT1; Artificial> (LCDR2-Antibody II)
YAVSKLDS
>SEQ ID NO: 6; PRT1; Artificial> (LCDR2-Antibody I)
YDVSKLDS
>SEQ ID NO: 7; PRT1; Artificial> (LCDR3-Antibody I and Antibody II)
VQGTHYPFT
>SEQ ID NO: 8; PRT1; Artificial> (HCVR-Antibody I and Antibody II)
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCTREGETVYWGQ
GTLVTVSS
>SEQ ID NO: 9; PRT1; Artificial> (LCVR-Antibody I)
DVVMTQSPLSLPVTLGQPASISCKSSQSLLYSRGKTYLNWFQQRPGQSPRRLIYD
VSKLDSGVPDRFSGSGSGTDFTLKISRVEAEDVGVYYCVQGTHYPFTFGQGTKLE
IK
>SEQ ID NO: 10; PRT1; Artificial> (LCVR-Antibody II)
DIQMTQSPSTLSASVGDRVTITCKSSQSLLYSRGKTYLNWLQQKPGKAPKLLIYA
VSKLDSGVPSRFSGSGSGTEFTLTISSLQPDDFATYYCVQGTHYPFTFGQGTKLEI
K
>SEQ ID NO: 11; PRT1; Artificial> (HC-Antibody I and Antibody II)
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCTREGETVYWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
>SEQ ID NO: 12; PRT1; Artificial> (LC-Antibody I)
DVVMTQSPLSLPVTLGQPASISCKSSQSLLYSRGKTYLNWFQQRPGQSPRRLIYD
VSKLDSGVPDRFSGSGSGTDFTLKISRVEAEDVGVYYCVQGTHYPFTFGQGTKLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
>SEQ ID NO: 13; PRT1; Artificial> (LC)-Antibody II)
DIQMTQSPSTLSASVGDRVTITCKSSQSLLYSRGKTYLNWLQQKPGKAPKLLIYA
VSKLDSGVPSRFSGSGSGTEFTLTISSLQPDDFATYYCVQGTHYPFTFGQGTKLEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
>SEQ ID NO: 14; DNA; Artificial> Exemplified DNA for Expressing
Antibody Heavy Chain of SEQ ID NO: 11
CAGGTGCAGCTGGTGCAGTCTGGGGCTGAGGTGAAGAAGCCTGGGTCCTCGG
TGAAGGTCTCCTGCAAGGCTTCTGGATACACCTTCACCGACTATTATATCAAC
TGGGTGCGACAGGCCCCTGGACAAGGGCTTGAGTGGATGGGATGGATCAACC
CTGGCAGTGGTAATACAAAGTACAATGAGAAGTTCAAGGGCAGAGTCACGAT
TACCGCGGACGAATCCACGAGCACAGCCTACATGGAGCTGAGCAGCCTGAGA
TCTGAGGACACGGCCGTGTATTACTGTACAAGAGAAGGCGAGACGGTCTACT
GGGGCCAGGGAACCCTGGTCACCGTCTCCTCAGCCTCCACCAAGGGCCCATC
GGTCTTCCCGCTAGCACCCTCCTCCAAGAGCACCTCTGGGGGCACAGCGGCCC
TGGGCTGCCTGGTCAAGGACTACTTCCCCGAACCGGTGACGGTGTCGTGGAA
CTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGG
ACAAGAAAGTTGAGCCCAAATCTTGTGACAAAACTCACACATGCCCACCGTG
CCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGT
GGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGACGAGCTGACCAAGAACCAGGTCAGCCTGACCTGCCT
GGTCAAAGGCTTCTATCCCAGCGACATCGCCGTGGAGTGGGAGAGCAATGGG
CAGCCGGAGAACAACTACAAGACCACGCCCCCCGTGCTGGACTCCGACGGCT
CCTTCTTCCTCTATAGCAAGCTCACCGTGGACAAGAGCAGGTGGCAGCAGGG
GAACGTCTTCTCATGCTCCGTGATGCATGAGGCTCTGCACAACCACTACACGC
AGAAGAGCCTCTCCCTGTCTCCGGGT
>SEQ ID NO: 15; DNA; Artificial> Exemplified DNA for Expressing
Antibody Light Chain of SEQ ID NO: 12
GATGTTGTGATGACTCAGTCTCCACTCTCCCTGCCCGTCACCCTTGGACAGCC
GGCCTCCATCTCCTGCAAGTCTAGTCAAAGCCTCCTGTACAGTCGCGGAAAAA
CCTACTTGAATTGGTTTCAGCAGAGGCCAGGCCAATCTCCAAGGCGCCTAATT
TATGATGTTTCTAAACTGGACTCTGGGGTCCCAGACAGATTCAGCGGCAGTGG
GTCAGGCACTGATTTCACACTGAAAATCAGCAGGGTGGAGGCTGAGGATGTT
GGGGTTTATTACTGCGTGCAAGGTACACACTACCCTTTCACTTTTGGCCAAGG
GACCAAGCTGGAGATCAAACGGACCGTGGCTGCACCATCTGTCTTCATCTTCC
CGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCC
TCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTC
ACAAAGAGCTTCAACAGGGGAGAGTGC
>SEQ ID NO: 16; DNA; Artificial> Exemplified DNA for Expressing
Antibody Light Chain of SEQ ID NO: 13
GACATCCAGATGACCCAGTCTCCTTCCACCCTGTCTGCATCTGTAGGAGACAG
AGTCACCATCACTTGCAAGTCCAGTCAGAGTCTCCTGTACAGTCGCGGAAAA
ACCTATTTGAACTGGCTCCAGCAGAAACCAGGGAAAGCCCCTAAGCTCCTGA
TCTATGCTGTCTCCAAACTGGACAGTGGGGTCCCATCAAGGTTCAGCGGCAGT
GGATCTGGGACAGAATTCACTCTCACCATCAGCAGCCTGCAGCCTGATGATTT
TGCAACTTATTACTGCGTGCAGGGTACACATTATCCTTTCACTTTTGGCCAGG
GGACCAAGCTGGAGATCAAACGGACCGTGGCTGCACCATCTGTCTTCATCTTC
CCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTC
ACAAAGAGCTTCAACAGGGGAGAGTGC
>SEQ ID NO: 17; PRT1; Artificial> (LCDR1-hE8L)
KSSQSLLYSRGKTYLN
>SEQ ID NO: 18; PRT1; Artificial> (LCDR2-hE8L)
AVSKLDS
>SEQ ID NO: 19; PRT1; Artificial> (LCDR3-hE8L)
VQGTHYPFT
>SEQ ID NO: 20; PRT1; Artificial> (HCDR1-hE8L)
GYTFTDYYIN
>SEQ ID NO: 21; PRT1; Artificial> (HCDR2-hE8L)
WINPGSGNTKYNEKFKG
>SEQ ID NO: 22; PRT1; Artificial> (HCDR3-hE8L)
EGETVY
>SEQ ID NO: 23; PRT1; Artificial> (LCVR-hE8L)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
K
>SEQ ID NO: 24; PRT1; Artificial> (HCVR-hE8L)
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCAREGETVYWGQ
GTTVTVSS
>SEQ ID NO: 25; PRT1; Artificial> (LC-hE8L)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
>SEQ ID NO, 26; PRT1; Artificial> (HC-hE8L)
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCAREGETVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
>SEQ ID NO: 27 PRT1; Artificial> N3pGlu Aβ
[pE]FRHDSGYEVHHQKLVFFAEDVGSNKGAIIGLMVGGVVIA
>SEQ ID NO: 28; PRT1; Artificial> (Aβ 1-42)
DAEFRHDSGYEVHHQKLVFFAEDVGSNKGAIIGLMVGGVVIA

1. A method of treating Alzheimer's disease in a patient, comprising administering to a patient in need of such treatment an effective amount of a compound of the formula: 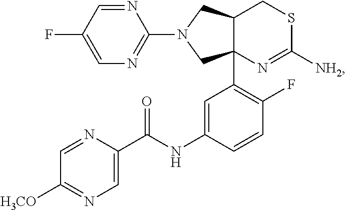 [SMILES: COc1cnc(C(=O)Nc2ccc(F)c([C@]34CN(c5ncc(F)cn5)C[C@H]3CSC(N)=N4)c2)cn1]
or a pharmaceutically acceptable salt thereof, in combination with an effective amount of an anti-N3pGlu Abeta antibody wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 21, and HCDR3 is SEQ ID. NO: 22
b) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3; and
c) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

2. The method according to claim 1 wherein the anti-N3pGlu Abeta antibody comprises a light chain variable region (LCVR) and a heavy chain variable region (HCVR), wherein said LCVR and HCVR are selected from the group consisting of:
a) LCVR of SEQ ID NO: 23 and HCVR of SEQ ID NO: 24
b) LCVR of SEQ ID NO: 9 and HCVR of SEQ ID NO: 8; and
c) LCVR of SEQ ID NO: 10 and HCVR of SEQ ID NO: 8.

3. The method according to claim 2 wherein the anti-N3pGlu Abeta antibody comprises a light chain (LC) and a heavy chain (HC), wherein said LC and said HC are selected from the group consisting of
a) LC of SEQ ID NO: 25 and HC of SEQ ID NO: 26;
b) LC of SEQ ID NO: 12 and HC of SEQ ID NO: 11; and
c) LC of SEQ ID NO: 13 and HC of SEQ ID NO: 11.

4. The method according to claim 3 wherein the anti-N3pGlu Abeta antibody comprises two light chains (LC) and two heavy chains (HC), wherein each LC and HC are selected from the group consisting of
a) LC of SEQ ID NO: 25 and HC of SEQ ID NO: 26;
b) LC of SEQ ID NO: 12 and HC of SEQ ID NO: 11; and
c) LC of SEQ ID NO: 13 and HC of SEQ ID NO: 11.

5. The method according to claim 2 wherein the LCVR is SEQ ID NO: 23 and the HCVR is SEQ ID NO: 24.

6. The method according to claim 3 wherein the LC is SEQ ID NO: 25 and the HC is SEQ ID NO: 26.

7. The method according to claim 4 wherein the LC is SEQ ID NO: 25 and the HC is SEQ ID NO: 26.

8. The method according to claim 1 wherein the compound is the tosylate salt of N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide.

9. The method according to claim 1 wherein the compound and the anti-N3pGlu Abeta antibody are administered simultaneously.

10. The method according to according to claim 1 wherein the compound is administered prior to the administration of the anti-N3pGlu Abeta antibody.

11. The method according to claim 1 wherein the anti-N3pGlu Abeta antibody is administered prior to the administration of the compound.

12. A pharmaceutical composition, comprising a compound of the formula: 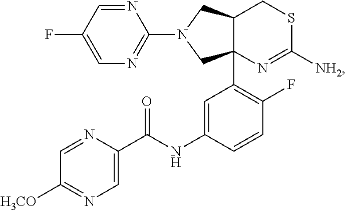 [SMILES: COc1cnc(C(=O)Nc2ccc(F)c([C@]34CN(c5ncc(F)cn5)C[C@H]3CSC(N)=N4)c2)cn1]
or a pharmaceutically acceptable salt thereof, with one or more pharmaceutically acceptable carriers, diluents, or excipients, in combination with a pharmaceutical composition of an anti-N3pGlu Abeta antibody wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 21, and HCDR3 is SEQ ID. NO: 22
b) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3; and
c) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3, with one or more pharmaceutically acceptable carriers, diluents, or excipients.

13. The pharmaceutical composition according to claim 12 wherein the anti-N3pGlu Abeta antibody comprises a light chain variable region (LCVR) and a heavy chain variable region (HCVR), wherein said LCVR and HCVR are selected from the group consisting of:
a) LCVR of SEQ ID NO: 23 and HCVR of SEQ ID NO: 24
b) LCVR of SEQ ID NO: 9 and HCVR of SEQ ID NO: 8; and
c) LCVR of SEQ ID NO: 10 and HCVR of SEQ ID NO: 8.

14. The pharmaceutical composition according to claim 13 wherein the anti-N3pGlu Abeta antibody comprises a light chain (LC) and a heavy chain (HC), wherein said LC and said HC are selected from the group consisting of
a) LC of SEQ ID NO: 25 and HC of SEQ ID NO: 26;
b) LC of SEQ ID NO: 12 and HC of SEQ ID NO: 11; and
c) LC of SEQ ID NO: 13 and HC of SEQ ID NO: 11.

15. The pharmaceutical composition according to claim 14 wherein the anti-N3pGlu Abeta antibody comprises two light chains (LC) and two heavy chains (HC), wherein each LC and each HC are selected from the group consisting of
a) LC of SEQ ID NO: 25 and HC of SEQ ID NO: 26;
b) LC of SEQ ID NO: 12 and HC of SEQ ID NO: 11; and
c) LC of SEQ ID NO: 13 and HC of SEQ ID NO: 11.

16. The pharmaceutical composition according to claim 13 wherein the LCVR is SEQ ID NO: 23 and the HCVR is SEQ ID NO: 24.

17. The pharmaceutical composition according to claim 14 wherein the LC is SEQ ID NO: 25 and the HC is SEQ ID NO: 26.

18. The pharmaceutical composition according to claim 12 wherein the compound is the tosylate salt of N-[3-[(4aR,7aS)-2-amino-6-(5-fluoropyrimidin-2-yl)-4,4a,5,7-tetrahydropyrrolo [3,4-d][1,3]thiazin-7a-yl]-4-fluoro-phenyl]-5-methoxy-pyrazine-2-carboxamide.

19. (canceled)

20. (canceled)

21. (canceled)

22. (canceled)

23. (canceled)

24. (canceled)

25. (canceled)


SEQUENCE LISTING (28 sequences)

SEQ ID NO: 1 - protein, 13 aa, Artificial Sequence
  Synthetic Construct
KASGYTFTDYYIN

SEQ ID NO: 2 - protein, 17 aa, Artificial Sequence
  Synthetic Construct
WINPGSGNTKYNEKFKG

SEQ ID NO: 3 - protein, 8 aa, Artificial Sequence
  Synthetic Construct
TREGETVY

SEQ ID NO: 4 - protein, 16 aa, Artificial Sequence
  Synthetic Construct
KSSQSLLYSRGKTYLN

SEQ ID NO: 5 - protein, 8 aa, Artificial Sequence
  Synthetic Construct
YAVSKLDS

SEQ ID NO: 6 - protein, 8 aa, Artificial Sequence
  Synthetic Construct
YDVSKLDS

SEQ ID NO: 7 - protein, 9 aa, Artificial Sequence
  Synthetic Construct
VQGTHYPFT

SEQ ID NO: 8 - protein, 115 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCTREGETVYWGQGTLVTVSS

SEQ ID NO: 9 - protein, 112 aa, Artificial Sequence
  Synthetic Construct
DVVMTQSPLSLPVTLGQPASISCKSSQSLLYSRGKTYLNWFQQRPGQSPRRLIYDVSKLDSGVPDRFSGSGSGTDFTLKISRVEAEDVGVYYCVQGTHYPFTFGQGTKLEIK

SEQ ID NO: 10 - protein, 112 aa, Artificial Sequence
  Synthetic Construct
DIQMTQSPSTLSASVGDRVTITCKSSQSLLYSRGKTYLNWLQQKPGKAPKLLIYAVSKLDSGVPSRFSGSGSGTEFTLTISSLQPDDFATYYCVQGTHYPFTFGQGTKLEIK

SEQ ID NO: 11 - protein, 444 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCTREGETVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 219 aa, Artificial Sequence
  Synthetic Construct
DVVMTQSPLSLPVTLGQPASISCKSSQSLLYSRGKTYLNWFQQRPGQSPRRLIYDVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 219 aa, Artificial Sequence
  Synthetic Construct
DIQMTQSPSTLSASVGDRVTITCKSSQSLLYSRGKTYLNWLQQKPGKAPKLLIYAVSKLDSGVPSRFSGSGSGTEFTLTISSLQPDDFATYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 1332 bp, Artificial Sequence
  Synthetic Construct
caggtgcagctggtgcagtctggggctgaggtgaagaagcctgggtcctcggtgaaggtctcctgcaaggcttctggatacaccttcaccgactattatatcaactgggtgcgacaggcccctggacaagggcttgagtggatgggatggatcaaccctggcagtggtaatacaaagtacaatgagaagttcaagggcagagtcacgattaccgcggacgaatccacgagcacagcctacatggagctgagcagcctgagatctgaggacacggccgtgtattactgtacaagagaaggcgagacggtctactggggccagggaaccctggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 15 - DNA, 657 bp, Artificial Sequence
  Synthetic Construct
gatgttgtgatgactcagtctccactctccctgcccgtcacccttggacagccggcctccatctcctgcaagtctagtcaaagcctcctgtacagtcgcggaaaaacctacttgaattggtttcagcagaggccaggccaatctccaaggcgcctaatttatgatgtttctaaactggactctggggtcccagacagattcagcggcagtgggtcaggcactgatttcacactgaaaatcagcagggtggaggctgaggatgttggggtttattactgcgtgcaaggtacacactaccctttcacttttggccaagggaccaagctggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 16 - DNA, 657 bp, Artificial Sequence
  Synthetic Construct
gacatccagatgacccagtctccttccaccctgtctgcatctgtaggagacagagtcaccatcacttgcaagtccagtcagagtctcctgtacagtcgcggaaaaacctatttgaactggctccagcagaaaccagggaaagcccctaagctcctgatctatgctgtctccaaactggacagtggggtcccatcaaggttcagcggcagtggatctgggacagaattcactctcaccatcagcagcctgcagcctgatgattttgcaacttattactgcgtgcagggtacacattatcctttcacttttggccaggggaccaagctggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 17 - protein, 16 aa, Artificial Sequence
  Synthetic Construct
KSSQSLLYSRGKTYLN

SEQ ID NO: 18 - protein, 7 aa, Artificial Sequence
  Synthetic Construct
AVSKLDS

SEQ ID NO: 19 - protein, 9 aa, Artificial Sequence
  Synthetic Construct
VQGTHYPFT

SEQ ID NO: 20 - protein, 10 aa, Artificial Sequence
  Synthetic Construct
GYTFTDYYIN

SEQ ID NO: 21 - protein, 17 aa, Artificial Sequence
  Synthetic Construct
WINPGSGNTKYNEKFKG

SEQ ID NO: 22 - protein, 6 aa, Artificial Sequence
  Synthetic Construct
EGETVY

SEQ ID NO: 23 - protein, 112 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 24 - protein, 115 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCAREGETVYWGQGTTVTVSS

SEQ ID NO: 25 - protein, 219 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCAREGETVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 40 aa, Artificial Sequence
  Synthetic Construct
  MISC_FEATURE (1)..(1): Xaa at position 1 = pyroglutamic acid
XFRHDSGYEVHHQKLVFFAEDVGSNKGAIIGLMVGGVVIA

SEQ ID NO: 28 - protein, 42 aa, Artificial Sequence [sequence omitted]